(19) United States
(12) Patent Application Publication
(10) Pub. No.: US 2016/0143726 A1
(43) Pub. Date: May 26, 2016

(54) Process for Preparing Tissue Regeneration Matrix

(71) Applicant: Integra LifeSciences Corporation, Plainsboro, NJ (US)

(72) Inventors: John E. Kemnitzer, West Windsor, NJ (US); Elaina Panas, Belle Mead, NJ (US)

(21) Appl. No.: 14/950,548

(22) Filed: Nov. 24, 2015

Related U.S. Application Data
(60) Provisional application No. 62/084,893, filed on Nov. 26, 2014.

(51) Int. Cl. A61F 2/10; A61L 27/44

(52) U.S. Cl. CPC A61F 2/105; A61L 27/44; A61F 2210/0004

(57) ABSTRACT

The present invention is directed to a process for making a tissue regeneration matrix. The process comprises providing a collagen-glycosaminoglycan-tropoelastin dispersion; freeze-drying the dispersion to provide a porous freeze-dried matrix; and then crosslinking the porous freeze-dried matrix. The present invention is also directed to a tissue regeneration matrix prepared by the process.

### RELATED APPLICATION DATA

This application claims priority to U.S. Provisional Patent Application No. 62/084,893, filed on Nov. 26, 2014 and is hereby incorporated herein by reference in its entirety for all purposes.

### FIELD OF THE INVENTION

The invention generally relates to the field of tissue regeneration. More specifically, the invention relates to tissue regeneration matrices comprising collagen for use in, for example, wound care and dermal regeneration, and processes for making the matrices.

### BACKGROUND

Collagen is a natural body material useful in a wide range of medical applications. The incorporation of glycosaminoglycans (GAG) into collagen is recognized as providing for a matrix that allows for regeneration of primary tissues. Thus, collagen/GAG matrices represent a particularly useful family of collagen-containing materials.

U.S. Pat. No. 4,947,840, which is incorporated herein by reference in its entirety, discloses a biodegradable polymeric material for treating wounds, which acts as a scaffold and induces the wound to synthesize new tissue. The material preferably comprises Type-I collagen and glycosaminoglycan (GAG) in a covalently crosslinked sheet. The material has been shown to reduce contraction and scarring of dermal wounds when used in a sheet form and placed over wounds to promote regeneration.

U.S. Pat. No. 6,969,523, which is incorporated herein by reference in its entirety, describes compositions of cross-linked collagen and a glycosaminoglycan which retain characteristics rendering them useful as tissue engineering matrices or scaffolds following terminal sterilization. These biodegradable matrices are useful in a variety of biochemical applications, including, but not limited to, dermal replacement constructs. For example, the dermal replacement layer of the Integra® Dermal Regeneration Template (Integra LifeSciences Corporation, Plainsboro, N.J., U.S.A.) is comprised of a porous matrix of fibers of cross-linked bovine tendon collagen and the glycosaminoglycan chondroitin-6-sulfate. This commercially available bilayer membrane system for skin replacement is useful in the treatment of deep, partial-thickness, or full-thickness thermal injury to the skin such as third-degree burns. Following application to the wound, the bilayer functions as an artificial skin that provides immediate post-excisional wound homeostasis, facilitating patient recovery and relieving metabolic stress.

Although some tissue regeneration materials are available commercially, there remains a need for tissue regeneration materials that have improved physical properties and effectiveness for tissue regeneration.

### SUMMARY

In accordance with an aspect of the present invention, a process for making tissue regeneration matrices is provided. The process comprises: a) providing a collagen-glycosaminoglycan-tropoelastin dispersion; (b) freeze-drying the collagen-glycosaminoglycan-tropoelastin dispersion to provide a porous freeze-dried matrix; and (c) crosslinking the porous freeze-dried matrix. The dispersion may comprise about 70 to about 95% collagen, about 2 to about 15% glycosaminoglycan, and about 2% to about 15% tropoelastin.

In accordance with another aspect of the present invention, a tissue regeneration matrix is provided. The tissue regeneration matrix is prepared by the process of the present invention described above.

In accordance with a further aspect of the present invention, a tissue regeneration matrix comprising collagen, glycosaminoglycan and elastin is provided, wherein the elastin is generated by crosslinking tropoelastin in the presence of collagen and glycosaminoglycan, in vitro. The matrix may further comprise tropoelastin.

In accordance with yet another aspect of the present invention, a tissue regeneration matrix comprising collagen, glycosaminoglycan and tropoelastin is provided. The tissue regeneration matrix is prepared by providing a mixture of collagen, glycosaminoglycan and tropoelastin, and crosslinking the mixture. The matrix may further comprise elastin.

These and other features and advantages of the invention or certain embodiments of the invention will be apparent to those skilled in the art from the following disclosure and description of exemplary embodiments.

### DETAILED DESCRIPTION OF EXEMPLARY EMBODIMENTS

The process of the present invention for preparing a tissue regeneration matrix comprises: a) providing a collagen-glycosaminoglycan-tropoelastin dispersion; (b) freeze-drying the collagen-glycosaminoglycan-tropoelastin dispersion to provide a freeze-dried matrix; and (c) crosslinking the freeze-dried matrix. The freeze-dried matrix may be porous, and it may remain porous after being cross-linked. The process may additionally comprise a step of dehydrothermal treatment of the porous freeze-dried matrix, and may further comprise a step of applying a synthetic polymeric layer, such as a silicone layer.

Embodiments of the present invention are based on the discovery that tropoelastin, for example, recombinant human tropoelastin (rhTE), may be solubilized and readily incorporated into a collagen-glycosaminoglycan (GAG) dispersion, presumably resulting in an ionic binding of the collagen-GAG dispersion. This dispersion is able to be lyophilized and dehydrothermally (DHT) processed resulting in a matrix with the appropriate porosity properties of a dermal regeneration matrix. This matrix may be further stabilized (i.e., crosslinked), without being bound by any theory, presumably locking in covalently the rhTE into the matrix structure. It is hypothesized that polymerization of the rhTE has also occurred during both the DHT and solution crosslinking steps. The resulting product may be sterilizable by E-beam irradiation.

Collagen is a major protein component of bone, cartilage, skin, and connective tissue in animals. Collagen occurs in several types, having differing physical properties. The most abundant types are Types I, II and III. In an exemplary embodiment of the present invention, Type I collagen is used in the process of the present invention.

Collagen derived from any source is suitable for use in the compositions of the present invention, including insoluble collagen, collagen soluble in acid, in neutral or basic aqueous solutions, as well as those collagens that are commercially available. Typical animal sources for collagen include but are not limited to recombinant collagen, fibrillar collagen from bovine, porcine, ovine, cuprine and avian sources as well as soluble collagen from sources such as cattle bones and rat tail tendon.

The term glycosaminoglycan or GAG describes hexosamine-containing polysaccharides. Another name often used for this class of compounds is mucopolysaccharides. Chemically, GAGs are alternating copolymers made up of residues of hexosamine glycosidically bound and alternating in a more or less regular manner with either hexuronic acid or hexose moieties. Various forms of glycosaminoglycans (GAG) which may be suitable for use in the process include, but are not limited to, hylauronic acid, chondroitin 6-sulfate, chondroitin 4-sulfate, heparin, heparin sulfate, keratin sulfate and dermatan sulfate. A preferred GAG for use in the present invention is chondroitin 6-sulfate. However, other GAG are suitable for forming the composite materials described herein, and those skilled in the art will either know or be able to ascertain, using no more than routine experimentation, other suitable GAG. For a more detailed description of GAG, see Aspinall, G. O., Polysaccharides, Pergamon Press, Oxford (1970).

Other types of molecules that can be used in combination with collagen during the manufacturing process include, but are not limited to, chitin, chitosan, fibronectin, laminin, decorin, and the like, or combinations thereof.

Elastin is an extracellular matrix protein that is found in connective tissues and other tissues such as skin. Elastin has elastic properties. Tropoelastin is the monomeric form of elastin. The monomer polypeptides form elastin when cross-linked. In vivo, tropoelastin monomers are cross-linked by lysyl oxidase to form elastin.

U.S. Pat. No. 6,808,707, which is incorporated herein by reference in its entirety, describes that the genes encoding tropoelastin have been cloned from a variety of organisms including human and non-human organisms, and that a variety of different isoforms of human tropoelastin are produced in nature (by alternative splicing). The gene and expression of human tropoelastin have been described in Indik et al (1990) Archives of Biochemistry and Biophysics, 280(1), 80-86; Martin et al. (1995) Gene, 154, 159-166; and U.S. Pat. Nos. 6,232,458 and 7,700,126, which are all incorporated herein by reference in their entireties. In addition, the tropoelastin protein may be modified, as compared with naturally occurring tropoelastin protein, either chemically or genetically in vivo or in vitro. Tropoelastin can be prepared recombinantly or by chemical synthesis. Tropoelastin in any form and from any source, including full length tropoelastin, isoforms of tropoelastin, genetically engineered tropoelastin constructs, fragments and derivatives of tropoelastin, or a combination thereof, may be used in the process of the present invention for preparing tissue generation matrices.

The collagen-glycosaminoglycan-tropoelastin dispersion comprises about 70 to about 95% collagen, about 2 to about 15% glycosaminoglycan, and about 2% to about 15% tropoelastin. In certain embodiments, the dispersion comprises about 50 to about 95% collagen, about 1 to about 25% glycosaminoglycan, and about 1% to about 25% tropoelastin, for example, about 80% to about 90% collagen, about 2% to about 10% glycosaminoglycan, and about 2% to about 12% tropoelastin.

For preparation of a collagen-glycosaminoglycan-tropoelastin dispersion, collagen, glycosaminoglycan, and tropoelastin may be added to the dispersion in any order. In exemplary embodiments, a collagen-glycosaminoglycan dispersion is prepared first, and then tropoelastin is manually mixed into the collagen-glycosaminoglycan dispersion to yield a collagen-glycosaminoglycan-tropoelastin dispersion, in a 90% collagen/10% tropoelastin (w/w) ratio (which equals about a 83% collagen/8% GAG/9% tropoelastin ratio), or a 97% collagen/3% tropoelastin (w/w) ratio (which equals about a 89% collagen/8% GAG/3% tropoelastin ratio). The freezing process was followed by sublimation of ice crystals to produce the scaffold pore structure. Dehydrothermal treatment was then performed at an elevated temperature, for example under vacuum pressure for a sufficient period of time.

As used herein for purposes of the present invention, the terms “matrix”, “matrices”, “scaffold” or “scaffolds” refer to a construct of natural or synthetic biomaterials, particularly collagens and their derivatives that can be used in a composite with a glycosaminoglycan (GAG) or other materials, which are used in vivo and in vitro as structural supports for cells and tissues, frameworks for tissue formation and regeneration, surfaces for cell contact, or delivery systems for therapeutics. Thus, by the term matrix or scaffold, it is meant to include load-bearing materials, bulking agent and fillers, and physiological barriers as well as frameworks for tissue formation and delivery systems for cells, biomolecules, drugs and derivatives thereof.

Covalent cross-linking can be achieved by various coupling or cross-linking reagents, some of which are suitable for biological applications. One suitable chemical method for covalently cross-linking collagen/GAG/tropoelastin matrices is known as aldehyde cross-linking. In this process, the materials are contacted with aqueous solutions of aldehyde, which serve to cross-link the materials. Suitable aldehydes include formaldehyde, glutaraldehyde and glyoxal. The preferred aldehyde is glutaraldehyde because it yields a desired level of cross-link density more rapidly than other aldehydes and is also capable of increasing the cross-link density to a relatively high level. When glutaraldehyde is used as the cross-linking agent, it is preferred that nontoxic concentrations of greater than about 0.25% be used. Other chemical techniques that are suitable for increasing cross-link density in the present invention include carbodiimide coupling, azide coupling, and diisocyanate cross-linking, as well as crosslinking with polyethylene glycol (PEG).

It is contemplated that the collagen-glycosaminoglycan-tropoelastin matrix scaffold may be in a single layer configuration, without a silicone layer, or in a multiple layer configurations, including one or more layers of biocompatible materials. In an exemplary embodiment, a collagen-glycosaminoglycan-tropoelastin matrix scaffold in a bilayer configuration, with a silicone layer, is provided for promoting dermal regeneration. The silicone layer may be applied to the collagen-glycosaminoglycan-tropoelastin matrix prior to cross-linking. This layer is applied in accordance with well-known techniques. An exemplary method for application of the silicone layer to the matrix or scaffold is set forth in Example 6. The silicone layer can be added prior to sterilization or after sterilization at the point of care.

The matrices of the invention can further comprise bioactive molecules effective to achieve a desired result. Suitable bioactive molecules include, but are not limited to, growth factors, anti-inflammatory agents, wound healing agents, anti-scarring agents, antimicrobial agents (for example, silver), cell-adhesion peptides including Arg-Gly-Asp (RGD) containing peptides, nucleic acids, nucleic acid analogues, proteins, peptides, amino acids, and the like, or combinations thereof.

Pharmacologically active agents such as VEGF (vascular endothelial cell growth factor), FGF (the fibroblast growth factor family of proteins), TGF.beta. (transforming growth factor B), hepatocyte growth factor (HGF), platelet factor 4 (PF4), PDGF (platelet derived growth factor), EGF (epidermal growth factor), NGF (nerve growth factor), BMP (bone morphogenetic protein family), coagulation factors such as one of the vitamin K-dependent coagulant factors, such as Factor II/IIa, Factor VIINIIa, Factor IX/IXa or Factor X/Xa. Factor V/Va, VIIINIIIa, Factor XI/XIa, Factor XII/XIIa, Factor XIII/XIIIa, and mixtures thereof may also be used. Antimicrobials, antibiotics, antifungal agents, hormones, enzymes, enzyme inhibitors, and mixtures thereof can also be incorporated in the compositions of the instant invention and subsequently delivered to the wound site.

Sterilization can be performed by any method conventional in the art, although electron beam irradiation is a preferred method, especially when a silicone layer is present.

The incorporation of tropoelastin into the collagen-GAG matrix provides improved properties and effectiveness compared to the original collagen-GAG matrix, including increased elasticity and mechanical properties, earlier revascularization and dermal regeneration, faster re-epithelization, and reduced scar and contracture. Pre-clinical evaluation appears to show that the tissue regeneration matrix prepared by the process of the present invention provides a superior matrix on several fronts in comparison to the collagen-GAG only matrix, or tropoelastin directly instilled onto the collagen-GAG matrix.

The collagen-glycosaminoglycan-tropoelastin matrix scaffold is porous and biodegradable. The average pore size is within the range of about 100 μm to about 600 μm. The pore volume is preferably within the range of 75-95%.

These matrices and scaffolds are useful in medical and surgical applications, for the regeneration of dermal and sub-dermal tissue. Specifically they can be used for dermal regeneration after excision of burns, scars, and other injuries or for filling of tissue, for example, after excisions of tumors or for cosmetic application such as augmentation of tissue. In this embodiment, the matrix or scaffold is applied to or implanted within a subject at or near the site of the excision or the site where augmentation of tissue is required. Methods for application of such matrices or scaffold are well known by those of skill in the art.

Other applications collagen-GAG-tropoelastin matrix scaffolds of the present invention may include, but are not limited to, surgical sutures, blood vessel grafts, catheters and, in general, the fabrication of surgical prostheses. Additionally, these matrices or scaffolds are useful in the fabrication of artificial organs that pump blood, such as artificial kidneys, and blood compatible equipment such as blood oxygenators, as well as in the fabrication of miscellaneous equipment for the handling and storage of blood such as pumps, tubes and storage bags.

### EXAMPLES

The following examples describe the manufacture of a collagen-glycosaminoglycan-tropoelastin matrix in a bilayer configuration with a silicone layer to promote dermal regeneration. The tropoelastin is added to the collagen-glycosaminoglycan (GAG) dispersion in a 90% collagen/10% tropoelastin (w/w) ratio (which equals about 83% collagen/8% GAG/9% tropoelastin), or 97% collagen/3% tropoelastin (w/w) ratio (which equals about 89% collagen/8% GAG/3% tropoelastin). The examples are specific embodiments of the present invention but are not intended to limit it.

### Example 1

### Preparation of Collagen-Glycosaminoglycan Dispersion

The collagen-glycosaminoglycan dispersion was produced consisting of Type I collagen (0.5% w/w) purified from bovine tendon (Integra Life Sciences, Plainsboro, New Jersey) and chondroitin-6-sulfate (0.05% w/w) (Seikagaku Corporation, Japan) in 0.05M acetic acid (Glacial Acetic Acid, USP, CAS No. 64-19-7) solution.

A 0.05M acetic acid solution was prepared by combining 50-55 kg of Water for Injection (WFI) with 330 g acetic acid, and continuously adding WFI while mixing to reach 110 kg (pH 3.2, 0.5-2° C.).

A glycosaminoglycan dispersion in 0.05M acetic acid was prepared by mixing 47.52 g chondroitin-6-sulfate (Seikagaku Corporation, Japan) into 18 kg 0.05M acetic acid and allowing it dissolve for a minimum of 1 hour, followed by stirring for a minimum of 30 minutes.

A collagen dispersion in 0.05M acetic acid was prepared by adding 500 g (dry weight) purified Type I collagen (Integra LifeSciences, Plainsboro, NJ) into 83.3 kg 0.05M acetic acid and mixing in a stainless steel vessel (9500 RPM) at 10° C. for 30 minutes.

Complexing of collagen to glycosaminoglycan was performed by slowly adding the 16.7 kg of glycosaminoglycan-acetic acid dispersion to the 83.8 kg collagen-acetic acid dispersion over the duration of 50 minutes while mixing at 9500 RPM and maintaining a temperature below 25° C.

After complete transfer, the collagen-glycosaminoglycan dispersion was mixed at 9500 RPM for an additional 60 minutes. The dispersion was then degassed for 30 minutes to remove air bubbles.

### Example 2

### Preparation of Collagen-Glycosaminoglycan-Tropoelastin Dispersion

GMP-grade recombinant human tropoelastin isoform SHELA26A (Synthetic Human Elastin without domain 26A) corresponding to amino acid residues 27-724 of GenBank entry AAC98394 (gi 182020) was provided by Elastagen Pty Ltd., and manually mixed into the collagen-glycosaminoglycan dispersion using a 90% collagen/10% tropoelastin (w/w) ratio to yield a dispersion.

A mass of 0.6434 g tropoelastin was manually mixed into 1156 g collagen-glycosaminoglycan dispersion and subsequently mixed by stir bar for 20 minutes at 20° C., then degassed for 30 minutes under vacuum to remove air bubbles.

A mass of 210 g of collagen-glycosaminoglycan-tropoelastin dispersion was pipetted into a stainless steel tray (inner tray dimensions 28.5 cm length×23.3 cm width×2.8 cm depth; Grade 316 SS) for lyophilization. The dispersion was freeze-dried (Millrock Magnum Max85 Freeze Dryer, Model MX85B10, Kingston, N.Y.) by first cooling from 10° C. to −35° C. at a rate of 0.25° C./min and then held at constant temperature for 2 hours.

### Example 3

### Lyophilization and Dehydrothermal Treatment

The freezing process was followed by sublimation of ice crystals to produce the scaffold pore structure. The sublimation process was performed at 200 mtorr vacuum pressure and under a temperature ramp rate of 0.83° C./min for the first 3 hours from −35° C. to −20° C., 0.013° C./min for 6.5 hours up to −15° C. and held at constant temperature for 1 hour, 0.03° C./min for the next 5 hours up to −5° C., and 0.125° C./min for 4 hours up to 25° C.

Dehydrothermal treatment was then performed at 105° C. under 200 mtorr vacuum pressure for 24 hours to generate dry collagen-glycosaminoglycan-tropoelastin matrix scaffolds.

### Example 4

### Crosslinking the Matrix

Chemical crosslinking was performed in a 0.5% (w/w) glutaraldehyde in 0.05M acetic acid solution.

Dry collagen-glycosaminoglycan-tropoelastin matrix scaffolds were soaked in 0.05M acetic acid (Glacial Acetic Acid, Cat No. 338826, Sigma-Aldrich, St. Louis, Mo.) followed by subsequent exposure to 0.5% glutaraldehyde (50% Glutaraldehyde solution, Cat No. 340855, Sigma-Aldrich, St. Louis, Mo.) in a 0.05M acetic acid solution and the performing a series of rinse steps to remove residual glutaraldehdye.

Dry scaffolds were lined single-sided with a polyethylene sheet for support, placed into meshed polypropylene frames and stacked horizontally in a Nalgene bin. A 0.05M acetic acid solution was prepared by adding 60 ml acetic acid to 19.94 L deionized water (pH 3.2; room temperature) and mixed for 30 minutes.

Using a peristaltic pump and tubing, the 0.05M acetic acid solution was pumped into the basin until scaffolds were completely submerged and soaked 17 hours. In a separate bin, the 0.5% glutaraldehyde crosslinking solution was prepared composed of 0.5% (w/w) glutaraldehyde in 0.05M acetic acid solution. Specifically, 198 ml glutaraldehyde solution (50%) was added to an acetic acid solution containing 60 ml acetic acid and 19.742 L deionized water and mixed by stir bar for 20 minutes.

Following the overnight soak, the acetic acid solution was removed from the original Nalgene bin using a peristaltic pump and replaced with the 0.5% glutaraldehyde crosslinking solution. Scaffolds were soaked in the glutaraldehyde crosslinking solution for 22.5 hours. Crosslinking solution was removed and replaced with deionized water for 30 minutes to rinse residual glutaraldehyde from scaffolds. Four additional rinse steps with deionized water were performed for 30 minutes each, with the final rinse lasting for 15 hours.

Following the final rinse, deionized water was removed and replaced with 10 mM sodium phosphate buffer solution (200 g 1M NaH₂PO₄·H₂O diluted in 19.8 L deionized water, followed by subsequent addition of 50 mL 1M NaOH, and incremental addition of 1M NaOH to achieve pH of 6.5) and soaked for 30 minutes prior to packaging.

### Example 5

### Sterilization

Sterilization was performed by electron beam irradiation (17.5-35 kGy).

Scaffolds were removed from mesh frames with polyethylene sheet, and sandwiched in between two polyethylene sheets to aid in product handling. Scaffolds were packaged in a foil moisture-barrier pouch and terminally sterilized by electron beam irradiation (17.5-25 kGy).

### Example 6

### Application of Silicone Layer

A silicone layer could be added prior to sterilization or after sterilization at the point of care. A 0.26″ silicone layer is added via feeding dry sponges or matrices through a silicone dispensing pump with silicone coated polyethylene sheets. Alternatively, a silicone layer may be added manually at the time of application.

Given the benefit of the above disclosure and description of exemplary embodiments, it will be apparent to those skilled in the art that numerous alternative and different embodiments are possible in keeping with the general principles of the invention disclosed here. Those skilled in this art will recognize that all such various modifications and alternative embodiments are within the true scope and spirit of the invention. The appended claims are intended to cover all such modifications and alternative embodiments. It should be understood that the use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc.

What is claimed is:

1. A process for making a tissue regeneration matrix, comprising:
a) providing a collagen-glycosaminoglycan-tropoelastin dispersion;
(b) freeze-drying the collagen-glycosaminoglycan-tropoelastin dispersion to provide a porous freeze-dried matrix; and
(c) crosslinking the porous freeze-dried matrix.

2. The process of claim 1, wherein the dispersion comprises about 50 to about 95% collagen, about 1 to about 25% glycosaminoglycan, and about 1% to about 25% tropoelastin.

3. The process of claim 1, wherein the dispersion comprises about 70 to about 95% collagen, about 2 to about 15% glycosaminoglycan, and about 2% to about 15% tropoelastin.

4. The process of claim 1, wherein the dispersion comprises about 80 to about 95% collagen, about 2 to about 10% glycosaminoglycan, and about 2% to about 12% tropoelastin.

5. The process of claim 1, wherein the crosslinking step is performed by subjecting the matrix to an aldehyde solution.

6. A tissue regeneration matrix prepared by the process of claim 1.

7. A tissue regeneration matrix comprising collagen, glycosaminoglycan, and elastin, wherein the elastin is generated by crosslinking tropoelastin in the presence of collagen and glycosaminoglycan in vitro.

8. A tissue regeneration matrix comprising collagen, glycosaminoglycan and tropoelastin, prepared by a) providing a mixture of collagen, glycosaminoglycan and tropoelastin, and b) crosslinking the mixture.
